(19) United States
(12) Patent Application Publication
(10) Pub. No.: US 2011/0306134 A1
(43) Pub. Date: Dec. 15, 2011

(54) RETENTION OF A STEM CELL PHENOTYPE

(76) Inventors: Matthew Dalby, Glasgow (GB); Nikolaj Gadegaard, Glasgow (GB); Richard Oreffo, Southampton (GB)

(21) Appl. No.: 13/138,476

(22) PCT Filed: Feb. 23, 2010
(86) PCT No.: PCT/GB2010/000327

§ 371 Date: Aug. 23, 2011

(30) Foreign Application Priority Data
Feb. 23, 2009 (GB) 0903040.4

(51) Int. Cl. C12N 5/074; C12N 5/0735; C12N 5/0797; C12M 1/22; C12M 1/00; C12N 5/071; C12N 5/0775; B82Y 99/00

(52) U.S. Cl. 435/395; 435/305.1; 977/778; 977/902

(57) ABSTRACT

A method for promoting the retention of a stem cell phenotype in a population of stem cells. A biocompatible substrate is provided, e.g. in the form of cultureware. The substrate has an arrangement of topographical features arrayed in a pattern based on a notional symmetrical lattice in which the distance between nearest neighbour notional lattice points is between 10 nm and 10 μm, and wherein the topographical features are either located in register with the respective notional lattice points or are locally misordered such that the centre of each topographical feature is at most 10% of the distance between nearest neighbour notional lattice points from its respective notional lattice point. A population of stem cells is provided in contact with said arrangement of topographical features. Culturing the population of stem cells under conditions that allow the stem cells to proliferate allows the retention of the stem cell phenotype in the population of stem cells.

### BACKGROUND TO THE INVENTION

1. Field of the Invention

The present invention relates to biocompatible substrates for promoting the retention of a stem cell phenotype, and methods of using these biocompatible substrates to promote the retention of a stem cell phenotype. The invention also relates to cultureware including such a biocompatible substrate for promoting the retention of a stem cell phenotype.

2. Related Art

References noted in abbreviated form in the body of the text are set out in full in the section at the end of the description headed “References”. The content of each reference is hereby incorporated by reference.

The use of stem cells will underpin regenerative medicine and is a key therapeutic target in preventative medicine through biochemical pathways. However, due to the pluripotent/multipotential nature of embryonic and adult stem cells, cell culture is time-consuming. Culture requires the continuous isolation of stem cells, or is compromised through the cells differentiating away into more mature cell types. For example, mesenchymal stem cells, which are adult stem cells found in the bone marrow and can form fat, ligament, tendon, bone, cartilage, nerve, endothelia and epithelia, have a default, spontaneous (often unwanted) differentiation into fibroblasts when unstimulated (Oreffo et al., 1998; Triffit et al., 1998; Mirmalek-Sani et al., 2006). Therefore, they have a limited “shelf-life” without chemical control. This spontaneous differentiation of stem cells in culture is a major barrier to research on stem cells, as the cells need to be isolated regularly (especially so for adult stem cells), which is time-consuming and expensive. To prevent the spontaneous default differentiation of stem cells into fibroblasts, chemically defined media, feeder layers and other materials are often used, which may influence cell behaviour and lead to modulations in biochemical signalling. Such problems further lead to poor reproducibility of results and artefactual observations.

It is known that the cellular microenvironment is important in the control of stem cell differentiation. For example, it has been shown that matrix elasticity can direct stem cell lineage specification of mesenchymal stem cells grown in culture (Engler et al., 2006). Specifically, it was found that soft matrices that mimic brain are neurogenic, stiffer matrices that mimic muscle are myogenic, and comparatively rigid matrices that mimic collagenous bone are osteogenic.

It has also been shown that substrates having a nanoscale topography can lead to an alteration of cellular function. For example, nanoscale topography has been shown to alter the functional behaviour of both adhesive (Sutherland et al, 2001) and connective tissue proteins (Denis et al, 2002).

Previously, the inventors have developed materials that are capable of promoting the differentiation of mesenchymal stem cells and osteoprogenitor cells into osteoblasts by providing a degree of misorder to the symmetry of a nanoscale topography (WO 2007/057693; Dalby et al., 2007; Gadegaard et al., 2008). Specifically, the inventors found that if mesenchymal stem cells were cultured on a substrate having a disordered lattice arrangement of topographical features, differentiation of these cells into osteoblasts could be achieved. However, mesenchymal stem cells cultured on a planar control substrate or on a substrate having a substantially ordered symmetrical lattice arrangement of topographical features were fibroblastic in appearance.

It has also been reported that culturing human mesenchymal stem cells on titanium dioxide (TiO₂) nanotubes with a diameter of about 70-100 nm can promote the differentiation of these stem cells into osteoblasts (Oh et al, 2009).

It has previously been shown that substrates printed with nanoscale ridges and grooves can help human embryonic stem cells keep their “sternness” better than human embryonic stem cells plated on standard flat culture surfaces (McFarlin et al., 2006). However, these human embryonic cells were only grown in culture for 5 days and the retention of a stem cell phenotype for cells cultured long term (i.e. longer than 5 days) was not demonstrated and is not predictable from the results of McFarlin et al., 2006.

### SUMMARY OF THE INVENTION

Surprisingly, in view of their earlier results, the present inventors have found that culturing stem cells on a on a substrate having a substantially ordered symmetrical lattice arrangement of topographical features may promote retention of stem cell phenotype.

Thus, in a general aspect, the present inventors have developed materials that are capable of promoting the retention of a stem cell phenotype by providing a nanoscale topography with a substantially ordered symmetry.

In a first preferred aspect, the present invention provides a method for promoting the retention of a stem cell phenotype in a population of stem cells, the method comprising the steps of:
(i) providing a biocompatible substrate having an arrangement of topographical features arrayed in a pattern based on a notional symmetrical lattice in which the distance between nearest neighbour notional lattice points is between 10 nm and 10 μm, and wherein the topographical features are either located in register with the respective notional lattice points or are locally misordered such that the centre of each topographical feature is at most 10% of the distance between nearest neighbour notional lattice points from its respective notional lattice point;
(ii) providing a population of stem cells in contact with said arrangement of topographical features; and
(iii) culturing the population of stem cells under conditions that allow the stem cells to proliferate.

Using this method, the present inventors have surprisingly shown that topographical features that are arrayed in a substantially symmetrical lattice pattern (i.e. a lattice pattern in which the centre of each topographical feature is at most 10% of the distance between nearest neighbour notional lattice points from its respective lattice point) unexpectedly promote the retention of a stem cell phenotype.

Preferred and/or optional features will now be set out. These are applicable singly or in any combination with any aspect of the invention, unless the context demands otherwise.

Preferably, the centre of each topographical feature is at most 10%, at most 5%, at most 4%, at most 3%, at most 2%, or at most 1% of the distance between nearest neighbour notional lattice points from its respective notional lattice point. For example, the centre of each topographical feature may be a distance of at most 50 nm, at most 40 nm, at most 30 nm, at most 20 nm, at most 15 nm, at most 10 nm, at most 5 nm, at most 4 nm, at most 3 nm, at most 2 nm, or at most 1 nm from its respective notional lattice point.

Preferably, for at least 50% of the topographical features, the centre of each topographical feature is at most 5% of the distance between nearest neighbour notional lattice points from its respective notional lattice point. More preferably, at least 60%, at least 70%, at least 80% or at least 90% of the topographical features satisfy this criterion, or any of the criteria in the preceding paragraph.

Preferably, the topographical features of the biocompatible substrate are recesses into and/or protrusions from the surface of the substrate. In particular, the topographical features may include pits. Additionally or alternatively, the topographical features may include upstanding pillars. Combinations of such features are also envisaged.

Preferably, the distance between nearest neighbour notional lattice points is at least 20 nm, at least 30 nm, at least 40 nm, at least 50 nm, at least 60 nm, at least 70 nm, at least 80 nm, at least 90 nm, at least 100 nm, at least 110 nm, at least 120 nm, at least 130 nm, at least 140 nm, at least 150 nm, at least 160 nm, at least 170 nm, at least 180 nm, at least 190 nm, at least 200 nm, at least 210 nm, at least 220 nm, at least 230 nm, at least 240 nm, at least 250 nm, at least 260 nm, at least 270 nm, at least 280 nm, at least 290 nm or about 300 nm. Preferably, the distance between nearest neighbour notional lattice points is at least 50 nm.

Preferably, the distance between nearest neighbour notional lattice points is at most 9 μm, at most 8 μm, at most 7 μm, at most 6 μm, at most 5 μm, at most 4 μm, at most 3 μm, at most 2 μm, at most 1 μm, at most 900 nm, at most 800 nm, at most 700 nm, at most 600 nm, at most 500 nm, or at most 400 nm. Preferably, the distance between nearest neighbour notional lattice points is at most 5 μm.

At present, the most preferred range for the distance between nearest neighbour notional lattice points is between 30 nm and 3 μm.

Preferably, the height or depth (e.g. the average maximum height or depth) of the topographical features is at least 5%, more preferably at least 10%, of the distance between nearest neighbour notional lattice points from the remainder of the surface of the substrate. For example, the height or depth of the topographical features may be at least 10 nm.

The topographical features may be cylindrical pits or pillars, cuboid pits or pillars, hemi-spherical pits or pillars, part-spherical pits or pillars, or another regular shape. Preferably, each topographical feature has a substantially identical shape.

Preferably, the diameter of the topographical features is at least 10%, more preferably at least 20%, at least 30%, at least 40% or at least 50%, of the distance between nearest neighbour notional lattice points. For example, the diameter of the topographical features may be at least 20 nm.

Preferably, within an area of the substrate within which an arrangement of topographical features is formed, the topographical features account for at most 50%, at most 40%, at most 30%, or at most 20% of the surface of the substrate. Preferably, within an area of the substrate within which an arrangement of topographical features is formed, the topographical features account for at least 5%, at least 10%, or at least 20% of the surface of the substrate. Most preferably, within an area of the substrate within which an arrangement of topographical features is formed, the topographical features account for between 5% and 35% of the surface of the substrate.

The nature of the symmetry on which the notional lattice is based may be selected from a parallelogram lattice, a rectangular lattice, a square lattice, a rhombic lattice, a trigonal lattice and a hexagonal lattice. Preferably, the notional lattice is either a rectangular lattice or a square lattice.

The substrate comprises a biocompatible material. Of particular interest here are polycarbonate, polymethylmethacrylate (PMMA), or poly ε-caprolactone (PCL). However, other biocompatible polymers may be used. Furthermore, other biocompatible materials such as metals and ceramics may also be used. Additionally or alternatively, the substrate may be formed of a biocompatible composite material, for example, in which a surface layer or layers is formed of one of the biocompatible materials mentioned above. In the case of ceramics, it is preferred to cast and sinter the ceramics rather than perform an embossing step, which is a preferred route for polymer materials.

Stem cells suitable for culture in the method of the present invention include any adult stem cells, such as mesenchymal stem cells, neural stem cells, haemopoietic stem cells, endothelial stem cells, or adipose-derived stem cells. Alternatively, embryonic stem cells may be cultured in the method of the present invention.

Preferably, the stem cells are cultured in step (c) for more than 5 days, more than days, more than 15 days, more than 20 days, more than 21 days, more than 25 days, more than 28 days, or more than 30 days.

The stem cells may be passaged at least once, at least twice, at least three times, at least four times, or at least five times during step (c).

Following step (c), the method may include the additional step of confirming that the stem cells have retained their stem cell phenotype. This can be done by testing the cells for the expression of stem cell markers, such as Stro-1, Alcam, human stem cell factor (HSCF), bone morphogenic protein receptor 1A (BMPR1A), Oct-4, Nanog and SOX2. Testing for the expression of stem cell markers may be carried out by any suitable method, such as quantitative RT-PCR, immunocytochemistry, oligoarray analysis, or fluorescent-activated cell sorting (FACS) analsyis.

In a second preferred aspect, the invention provides cultureware for promoting the retention of a stem cell phenotype, said cultureware including a biocompatible substrate having an arrangement of topographical features arrayed in a pattern based on a notional symmetrical lattice in which the distance between nearest neighbour notional lattice points is between 10 nm and 10 μm, and wherein the topographical features are either located in register with the respective notional lattice points or locally misordered such that the centre of each topographical feature is at most 10% of the distance between nearest neighbour notional lattice points from its respective notional lattice point.

Suitable preferred and/or optional features have been set out above.

Cultureware of the present invention is preferably suitable for the culture of any adult stem cell, such as mesenchymal stem cells, neural stem cells, haemopoietic stem cells, endothelial stem cells, or adipose-derived stem cells, or of embryonic stem cells.

Cultureware of the present invention may include culture flasks, petri dishes, plates and coverslips.

Cultureware of the present invention may optionally have a population of stem cells stored on its biocompatible surface. For example, the cultureware may be a cell seeded construct or conduit.

### BRIEF DESCRIPTION OF THE DRAWINGS

Specific embodiments of the invention will now be described, by way of example, with reference to the accompanying drawings, in which:

FIG. 1 shows a schematic plan view of a nanotopography according to an embodiment of the invention.

FIG. 2 shows an SEM micrograph of a nanotopography according to an embodiment of the invention.

FIG. 3 is a histogram showing the variation in the centre-to-centre spacing between neighbouring topographical features of a square lattice designed to be a perfectly ordered square lattice with a distance of 300 nm between neighbouring notional lattice points.

FIG. 4 shows SEM images of various nanotopographies according to embodiments of the invention. The diameter of the topographical features decreases progressively along panels A to Q, while the distance between nearest neighbouring notional lattice points remains constant. For example, in panel E the diameter of each nanopit is 188 nm and the distance between the centres of neighbouring nanopits is 300 nm (giving 31% surface coverage by the nanopits), in panel P the diameter of each nanopit is 97 nm and the distance between the centres of neighbouring nanopits is 300 nm (giving 8% surface coverage by the nanopits), and in panel Q the diameter of each nanopit is 68 nm and the distance between the centres of neighbouring nanopits is 300 nm (giving 4% surface coverage by the nanopits), as indicated in the drawing.

FIG. 5 shows light microscope images of human fibroblast cells grown on each of the nanotopograhpies shown in FIG. 3. The cells were cultured for (A) 3 h, (B) 24 h, or (C) 72 h and stained with Coomassie blue.

FIG. 6 shows actin staining of human mesenchymal stem cells (HMSCs) cultured on a planar control PMMA substrate. Cells were cultured for (A, D, G and J) 14 days, (B, E, H and K) 21 days, and (C, F, I and L) 28 days.

FIG. 7 shows (A-C) osteopontin (OPN), (D-F) osteocalcin (OCN), (G-I) Alcam and (J-L) Stro-1 staining of human mesenchymal stem cells (HMSCs) cultured on a planar control PMMA substrate. Cells were cultured for (A, D, G and J) 14 days, (B, E, H and K) 21 days, and (C, F, I and L) 28 days. Samples correspond to those shown in FIG. 6.

FIG. 8 shows actin staining of human mesenchymal stem cells (HMSCs) grown on a PMMA substrate having a square (SQ) array of pits. Cells were cultured for (A, D, G and J) 14 days, (B, E, H and K) 21 days, and (C, F, I and L) 28 days.

FIG. 9 shows (A-C) osteopontin (OPN), (D-F) osteocalcin (OCN), (G-I) Alcam and (J-L) Stro-1 staining of human mesenchymal stem cells (HMSCs) grown on a PMMA substrate having a square (SQ) array of pits. Cells were cultured for (A, D, G and J) 14 days, (B, E, H and K) 21 days, and (C, F, I and L) 28 days. Samples correspond to those shown in FIG. 8.

FIG. 10 shows (A-C) osteopontin (OPN), (D-F) osteocalcin (OCN), (G-I) Alcam and (J-L) Stro-1 staining of human mesenchymal stem cells (HMSCs) grown for 7 days on (A, D, G and J) a PCL substrate having a square (SQ) array of pits, (B, E, H and K) a planar control PCL substrate, and (C, F, I and L) a planar control PCL substrate in the presence of 10 mM dexamethasone (DMX) and 150 pg/ml L-ascorbic acid.

FIG. 11 shows (A-C) osteopontin (OPN), (D-F) osteocalcin (OCN), (G-I) Alcam and (J-L) Stro-1 staining of human mesenchymal stem cells (HMSCs) grown for 21 days on (A, D, G and J) a PCL substrate having a square (SQ) array of pits, (B, E, H and K) a planar control PCL substrate, and (C, F, I and L) a planar control PCL substrate in the presence of 10 mM dexamethasone (DMX) and 150 pg/ml L-ascorbic acid.

FIG. 12 shows (A-C) osteopontin (OPN), (D-F) osteocalcin (OCN), (G-I) Alcam and (J-L) Stro-1 staining of human mesenchymal stem cells (HMSCs) grown for 28 days on (A, D, G and J) a PCL substrate having a disordered square array of pits ±50 nm (NSQ50), (B, E, H and K) a planar control PCL substrate, and (C, F, I and L) a planar control PCL substrate in the presence of 10 mM dexamethasone (DMX) and 150 pg/ml L-ascorbic acid.

### DETAILED DESCRIPTION OF THE PREFERRED EMBODIMENTS, AND FURTHER PREFERRED FEATURES OF THE INVENTION

First will be described the methods employed to manufacture biocompatible substrates according to embodiments of the invention, and alternative biocompatible substrates for comparison. Then will be described the particular experimental tests carried out on the various substrates, with a discussion of the technical significance of the results.

### Manufacture of Biocompatible Substrates

Suitable patterns having a desired array topographical features are produced in a master. The desired patterns may include a limited amount of local misorder. In the method used to obtain the experimental results presented herein, the master is formed of silicon, since patterning of silicon is well-understood. The silicon master is near atomically flat before patterning and is sufficiently conducting during the electron exposure to avoid sample charging. The desired pattern is generated by a computer program in which a suitable notional lattice is defined and each topographic feature is either placed in register with the respective notional lattice points, or locally misordered along the axes of the lattice by a random, limited amount. The software generates a file suitable for an electron beam lithography tool to read and execute. The silicon substrate is coated with a polymeric material, generally termed resist, which is susceptible to electron exposure. In the regions where the electron beam lithography tool exposes the resist, the regions will either be removed or left behind after development. This is determined by the type of resist used, generally termed positive or negative resist. Such considerations as the nature of the resist and the nature of the substrate will be well understood by a person skilled in the art.

Known suitable electron beam lithography tools have a grid resolution of 5 nm. Recently, more advanced electron beam lithography tools have become available that have a grid resolution of 0.5 nm. Suitable electron beam lithography tools will be known to persons skilled in the art. The resolution of the position of the topographic features is determined by the grid resolution of the electron beam lithography tool. However, there is also a stochastic displacement as a result of signal noise, temperature variations etc.

After patterning of the resist on the surface of the silicon, there are at least two options for forming a biocompatible polymeric substrate. For prototyping, the pattern formed in the resist can be transferred to the silicon through a reactive ion etch process. This yields a silicon surfaces with a topographic pattern which can be transferred by embossing to a suitable polymeric material. Alternatively, a nickel shim can be formed from the master structure by electro plating, a process well-known and used in the optical storage industry (CDs and DVDs). To make a nickel shim the master structure is first coated with a thin conducting metal film which subsequently acts as an electrode during the galvanic electroplating. The formed nickel shim is a negative copy of the master structure and can be used to make biocompatible replicas by embossing or injection moulding.

The substrate comprises a biocompatible material. Of particular interest here are polycarbonate, polymethylmethacrylate (PMMA), or poly ε-caprolactone (PCL).

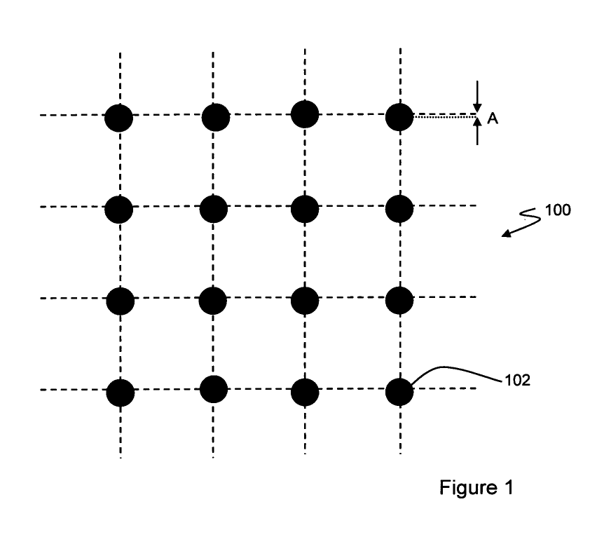

FIG. 1 shows a schematic plan view of a nanotopography 100 formed from nanopits 102, based on a notional square lattice (the notional lattice points being defined by the intersections of straight dashed lines). As can be seen from this drawing, the nanopits 102 are offset to a limited degree from their respective notional lattice points by a distance labelled as A. Note that it is possible to specify that the degree of misorder along one axis is different to the degree of misorder along another axis of the notional lattice.

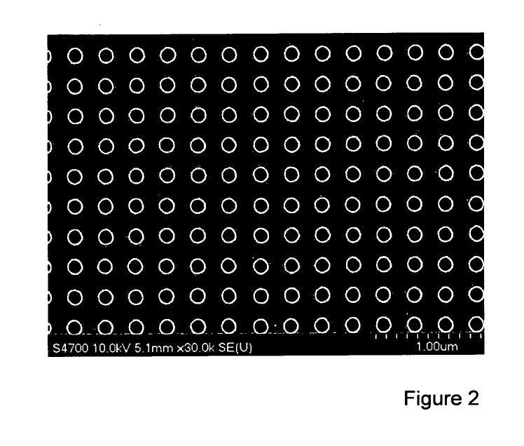

FIG. 2 shows an SEM micrograph of a square array of 120 nm diameter pits 100 nm deep with 300 nm centre-to-centre spacing. Although the square lattice used was designed to be perfectly ordered, given the limit on the grid resolution of the electron beam lithography tool used, each nanopit was up to approximately 5 nm from its respective notional lattice point. Therefore, the square lattice used has a small, but controlled, degree of local misorder.

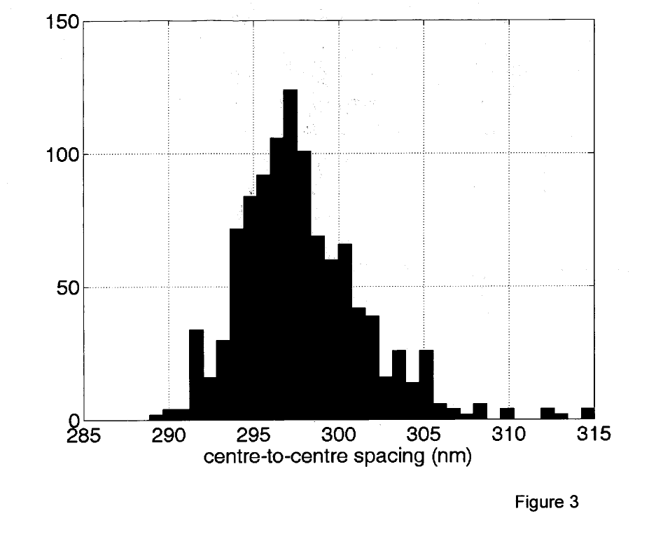

FIG. 3 is a histogram showing the variation in the centre-to-centre spacing between neighbouring topographical features of a square lattice designed to have a distance of 300 nm between neighbouring notional lattice points. This shows that the square array of topographical features did not have “perfect”, but that the array had a small, controlled degree of local misorder of about +/−2 nm.

### Effect of Surface Coverage by Nanopits on Fibroblast Growth and Adhesion

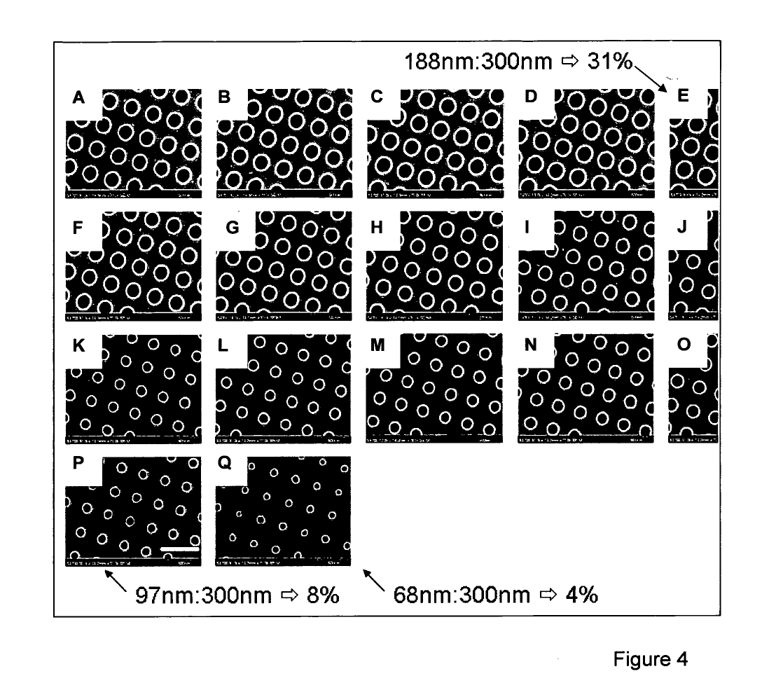

A pattern library of square nanotopograhies having areas with varying degrees of surface coverage by the topographical features was produced, such that within each area of the substrate within which an arrangement of topographical features was formed, the topographical features accounted a different percentage of the surface of the substrate. This pattern library is shown in FIG. 4. In each of the panels shown in FIG. 4, the topographical features (nanopits in this case) are substantially located in register with the respective notional lattice points of a square lattice. The diameter of the nanopits decreases sequentially along the panels labelled A-Q, such that in panels E, P and Q, the diameter of the nanopits is 188 nm, 97 nm and 68 nm respectively. In all panels, the distance between the nearest neighbour notional lattice points is 300 nm. Therefore, the percentage surface coverage of the substrate by the nanopits decreases sequentially along the panels labelled A-Q, such that in the panels indicated, the surface coverage is 31% (in panel E), 8% (in panel P) and 4% (in panel Q) respectively.

In order to assess the effect of surface coverage by nanopits on cell growth and adhesion, human fibroblast cells (h-TERT) were cultured on this pattern library for 3 h, 24 h and 72 h. The cells were then fixed, stained with Coomassie blue and visualised using light microscopy.

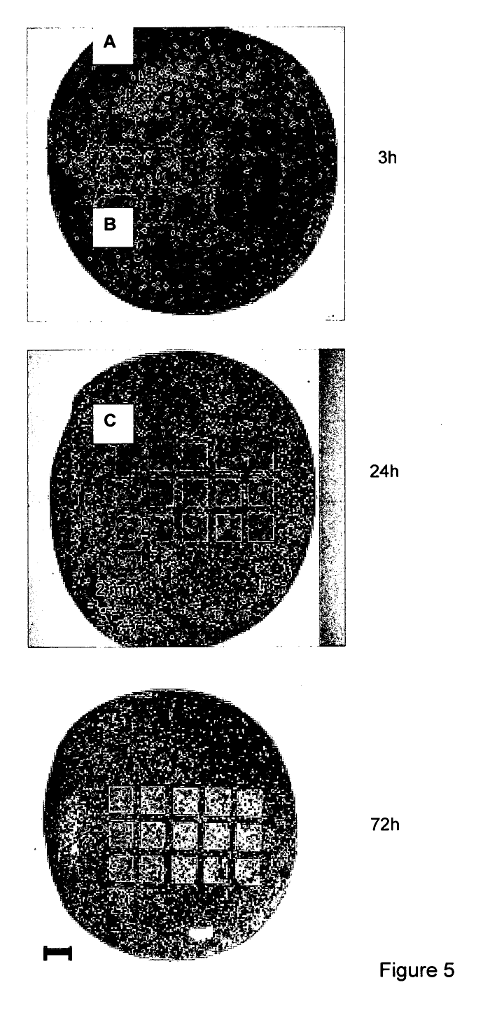

As can be seen from FIG. 5, the amount of cell growth and adhesion increased over time. However, after 24 h and 72 h, cell growth and cell adhesion was less good when the surface coverage by the nanopits is less than 10% than when it is greater than 10%.

In order to assess the effect of the prepared biocompatible substrates on stem cell phenotype, the following tests were carried out.

### Effect of Surface Nantopography on Differentiation of Human Mesenchymal Stem Cells (HMSCs)

Immunocytochemical analysis was carried out on primary HMSCs cultured on an ordered square lattice of topographical features formed on a poly ε-caprolactone or PMMA substrate.

HMSCs can give rise to cells of the adipogenic (fat), chondrogenic (cartilage), osteoblastic (bone), myoblastic (muscle) and fibroblastic and reticular (connective tissue) lineages and generate intermediate progenitors with a degree of plasticity. Thus, HMSCs give rise to a hierarchy of bone cell populations with a number of developmental stages: mesenchymal stem cells (MSCs), determined osteoprogenitor cells, preosteoblasts, osteoblasts and, ultimately, osteocytes.

Very immature, purely stem cell populations of HMSCs were isolated from bone marrow by FACS using an anti-Stro-1 antibody. Cultures were maintained in basal media (α-MEM containing 10% FCS) at 37° C., supplemented with 5% CO₂. All studies were conducted using passage 1 and passage 2 cells.

The HMSCs were seeded onto the test substrates at a density of 1×10⁴ cells per sample in 1 ml of complete medium. The medium used was α-MEM with 10% FCS (Life Technologies, UK). The cells were incubated at 37° C. with a 5% CO₂ atmosphere for 14, 21 or 28 days and the medium was changed twice a week.

At 14, 21 and 28 days, the cells were formaldehyde fixed for fluorescence or gluteraldehyde fixed for SEM. For SEM, the cells were next post-fixed in osmium tetroxide and dehydrated through a graded series of alcohols before air-drying with HMDS, gold coating and viewing. For fluorescence, cells were permeabilised with triton X and then stained with phalloidin-rhodamine to stain actin, with antibodies for Alcam and Stro-1 (which are stem cell-specific marker proteins), and with antibodies to the osteoblast-specific extracellular matrix proteins osteocalcin (OCN) and osteopontin (OPN). Secondary antibodies were then used to conjugate fluoroscein to the primary antibodies.

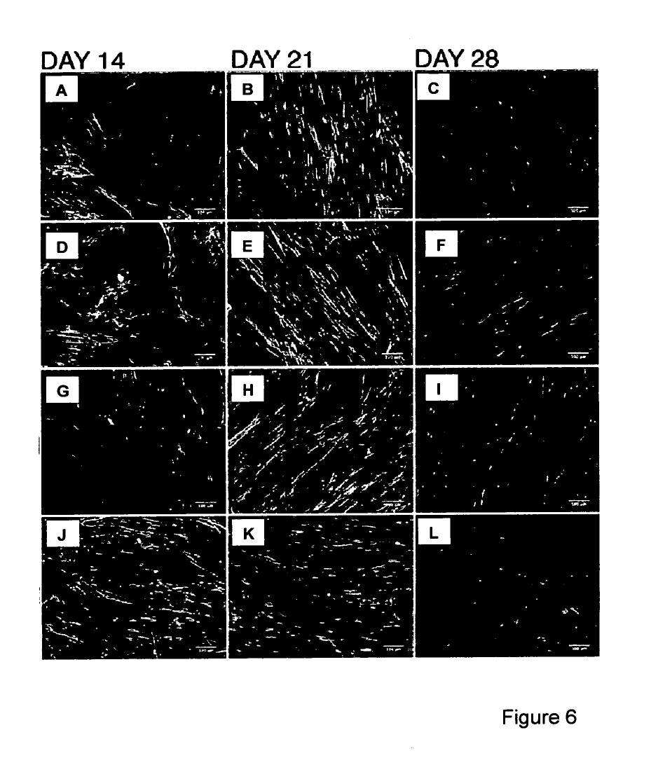

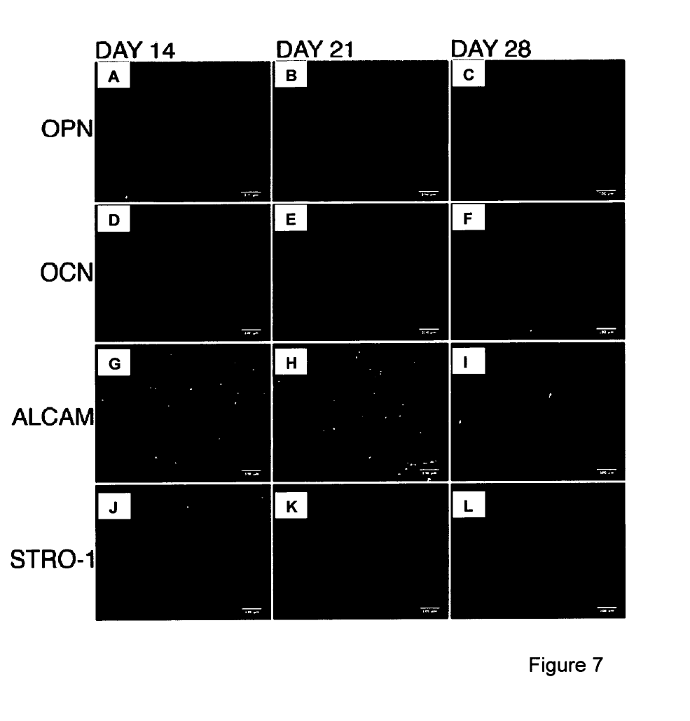

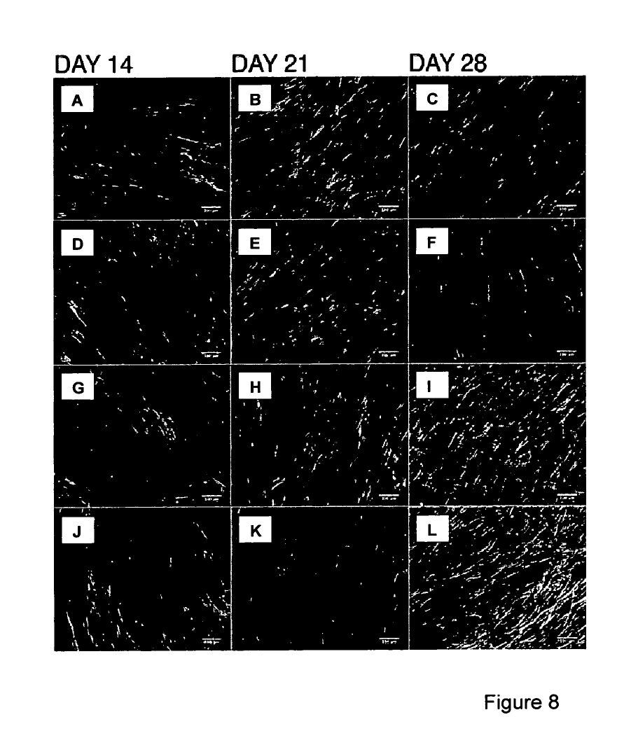

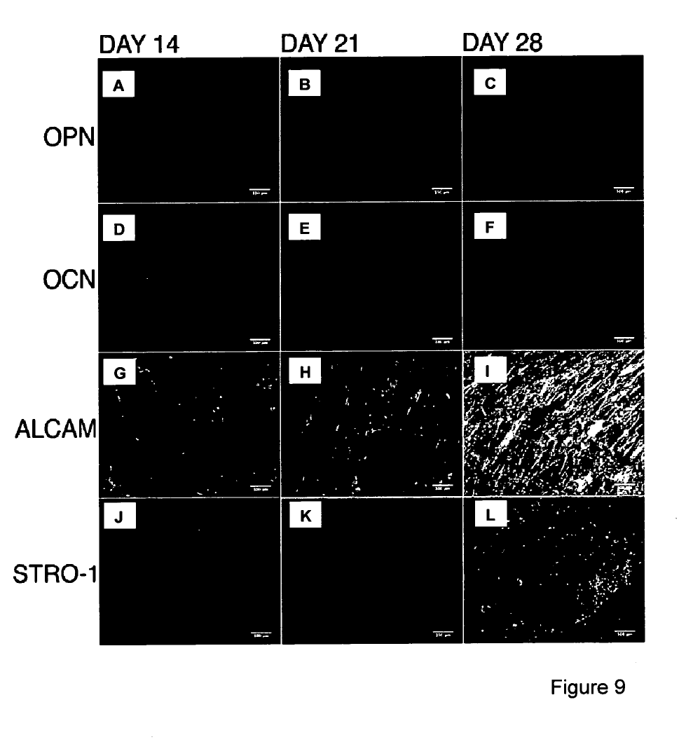

FIG. 6 shows actin staining for HMSCs cultured on a planar control PMMA substrate and FIG. 7 shows staining for the stem cell-specific proteins Stro-1 and Alcam and the osteoblastic ECM proteins OPN and OCN, also for HMSCs cultured on a planar control PMMA substrate. FIG. 8 shows actin staining for HMSCs cultured on a PMMA substrate having a square array of 120 nm diameter pits 100 nm deep nanopits with 300 nm centre-to-centre spacing, and FIG. 9 shows staining for the stem cell-specific proteins Stro-1 and Alcam and the osteoblastic ECM proteins OPN and OCN, also for HMSCs cultured on a PMMA substrate having a square array of 120 nm diameter pits 100 nm deep nanopits with 300 nm centre-to-centre spacing. Although this square lattice was designed to be perfectly ordered, given the limit on the grid resolution of the electron beam lithography tool used, each nanopit was up to approximately 2 nm from its respective notional lattice point. Therefore, the square lattice used has a small, controlled, degree of local misorder, as shown in FIG. 3.

Neglibible OPN, OCN, and Stro-1 staining, and minimal Alcam staining, was observed for the HMSCs grown on the planar control substrate after 14, 21 or 28 days in culture (see FIG. 7). Morphological analysis showed that these cells appeared to have differentiated into fibroblasts, which is the spontaneous default pathway for HMSCs grown long term in culture (see FIG. 6).

However, HMSCs cultured on the substantially ordered square lattice stained positively for Alcam, with very intense staining being observed after 28 days (see FIG. 91). Similarly, strong Stro-1 staining was observed for HMSCs cultured on the square lattice for 28 days (see FIG. 9L). Therefore, HMSCs grown on a substrate with a substantially ordered square lattice of nanopits retained their stem cell characteristics. As shown in FIG. 9, their stem cell phenotype actually appeared to be enhanced with time. This is surprising, as it had previously been thought that HMSCs grown on an ordered lattice arrangement of topographical features differentiated into fibroblasts, thus losing their stem cell phenotype (WO 2007/057693). Therefore, while HMSCs grown on a disordered symmetrical lattice of nanotopographical features differentiate into osteoblasts (WO 2007/057693; Dalby et al., 2007; Gadegaard at al., 2008), HMSCs grown on a substantially ordered symmetrical lattice retain their stem cell phenotype.

Therefore, using a substantially ordered lattice arrangement of nanotopographical features, it is possible to promote the retention of a stem cell phenotype without the use of chemical intervention for stem cells grown in long term cultures.

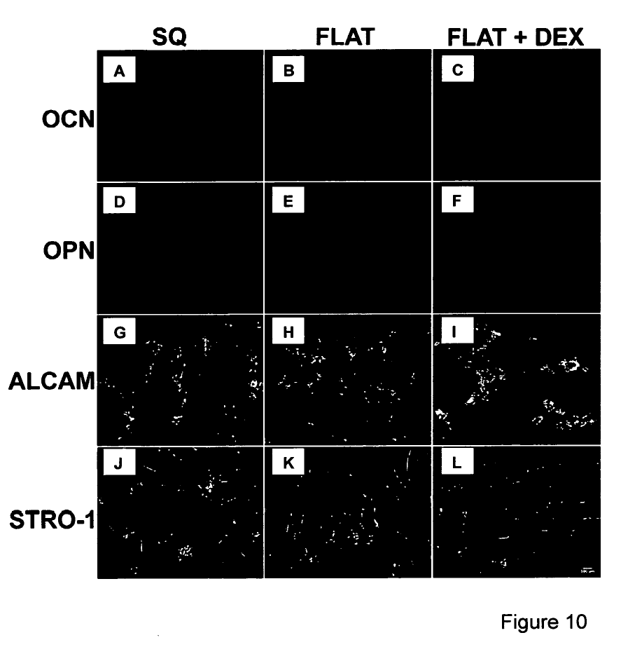

FIG. 10 shows staining for the stem cell-specific proteins Stro-1 and Alcam and the osteoblastic ECM proteins OPN and OCN of HMSCs cultured for 7 days on a PCL substrate having square array of 120 nm diameter pits 100 nm deep nanopits with 300 nm centre-to-centre spacing, or on a planar control PCL substrate in the presence or absence of dexamethasone (DMX), which is a corticosteroid that can induce bone formation. Data shown in FIG. 10 show that after 7 days in culture, the HMSCs grown on each of the substrates have a stem cell phenotype (i.e. they express the stem cell markers Alcam and Stro-1) and do not have any osteoblastic characteristics. Therefore, these data confirm that HMSCs grown on each substrate have a stem cell phenotype at the start of the culture period.

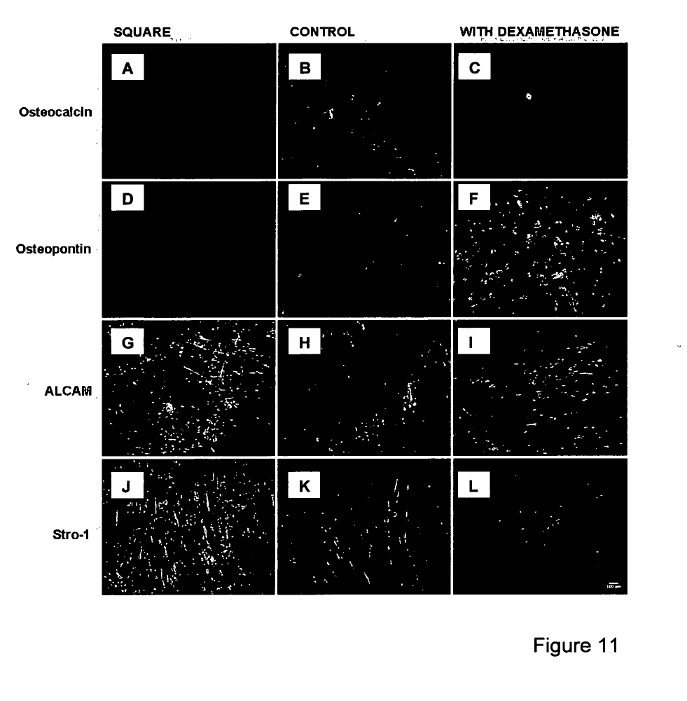

FIG. 11 shows that after 28 days in culture, HMSCs grown on a planar control PCL substrate in the presence of DMX express the osteoblastic markers osteocalcin and osteopontin (i.e. bone formation is stimulated). The HMSCs grown on the planar control had become a mixed cell population with some bone characteristics (i.e. some osteocalcin and osteopontin staining) and some stem cell characteristics (i.e. some Alcam and Stro-1 staining). In contrast, the HMSCs cultured on the square array of nanopits retained their stem cell characteristics and stained strongly for Alcam and Stro-1, while not expressing the osteoblastic markers osteocalcin and osteopontin. Therefore, the HMSCs grown on the square array were still a very pure stem cell population even after 28 days in culture.

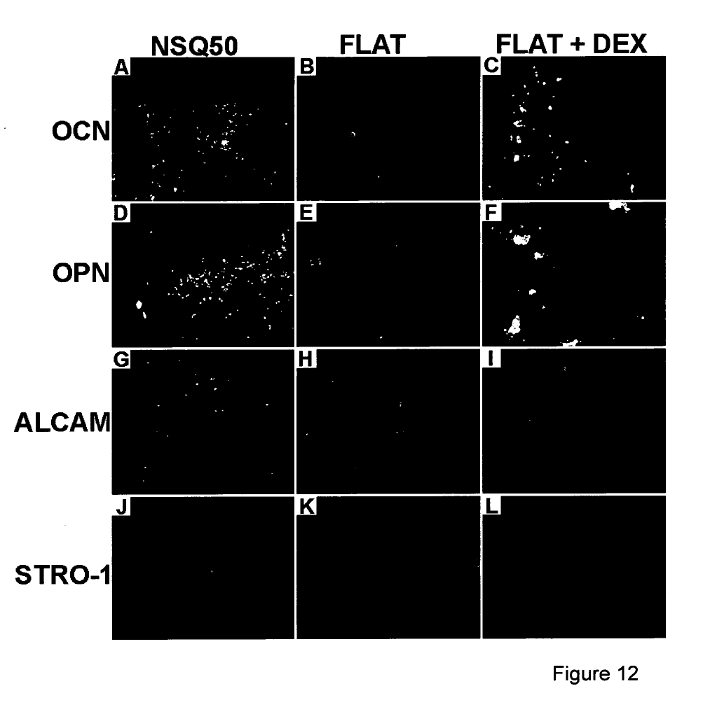

To assess whether or not HMSCs grown on a disordered square lattice retained their stem cell phenotype, HMSCs were cultured on a PCL substrate having a disordered square array of nanopits ±50 nm, i.e. the centre of each nanopit was approximately 50 nm from its respective notional lattice point. Staining for the stem cell-specific proteins Stro-1 and Alcam and the osteoblastic ECM proteins OPN and OCN showed that while cells grown on a disordered lattice arrangement expressed the osteoblastic cell markers osteocalcin and osteopontin, there was negligible expression of the stem cell markers Alcam and Stro-1 (see FIG. 12, panels A, D, G and J). Therefore, while stem cells grown on a square lattice with a small, controlled, degree of local misorder retained their stem cell phenotype in culture, stem cells grown on a more disordered square lattice differentiated into osteoblasts and lost their stem cell characteristics. Consistent with the results shown in FIG. 7, HMSCs grown on a planar control surface showed negligible expression of the osteoblast markers osteocalcin and osteopontin and of the stem cell markers Alcam and Stro-1 (see FIG. 12, panels B, E, H and K). However, treatment of the cells with DMX promoted the differentiation of these cells into osteoblasts (see FIG. 12, panels C, F, I and L).

Stem Cell-Specific Oligo Array Analysis of HMSCs Cultures on Substrates with Different Surface Nanotopographies

Stem cell-specific oligo array analysis using microarrays containing 101 genes specific for stem cells showed that after 21 days of culture, (i) HMSCs grown on planar control substrates expressed 18 stem cells genes, (ii) HMSCs grown on a substantially ordered square lattice of nanopits expressed 24 stem cell genes, and (iii) HMSCs grown on the disordered nanotopography (NSQ50) expressed just 1 stem cell related gene. These results (see Table 1) confirmed that HMSCs cultured on a substantially ordered square lattice of topographical features retained their stem cell phenotype better than HMSCs grown on a planar control lattice, while HMSCs grown on a disordered square lattice lost their stem cell phenotype.

Table 1 below shows the expression of particular stem cell specific genes by HMSCs cultured for 21 days on a planar control PMMA substrate, on a PMMA substrate having a square (SQ) array of pits, and on a PMMA substrate having a disordered square array of pits ±50 nm (NSQ50).

TABLE 1
Expression of stem cell specific genes by HMSC
cultured on substrates with different surface
nanotopographies

| Gene | Name | Control | Square | NSQ50 | Function |
|---|---|---|---|---|---|
| ABCG2 | ATP binding | X | X | | ABC proteins transport |
| | cassette G2 | | | | molecules across extra- and |
| | | | | | intracellular membranes |
| ALPPL2 | Alkaline | X | X | X | Produces free phosphate |
| | phosphatase, | | | | |
| | placental-like 2 | | | | |
| BAMBI | BMP and avidin | X | X | | Developmental pathways |
| | membrane bond | | | | |
| | inhibitor | | | | |
| BMP4 | Bone | X | X | | Cellular differentiation |
| | morphogenic | | | | |
| | protein 4 | | | | |
| BMPR2 | Bone | X | X | | Cellular differentiation |
| | morphogenic | | | | |
| | protein receptor, | | | | |
| | type II | | | | |
| CD4 | CD4 | | X | | T-cell binding |
| CDC42 | Cell division | | X | | The protein encoded by this |
| | cycle 42 | | | | gene is a small GTPase of the |
| | | | | | Rho-subfamily, which |
| | | | | | regulates signaling pathways |
| | | | | | that control diverse cellular |
| | | | | | functions including cell |
| | | | | | morphology, migration, |
| | | | | | endocytosis and cell cycle |
| | | | | | progression |
| CDH12 | Cadherin 12, | X | X | | Calcium dependent cell-cell |
| | type 2 | | | | adhesion |
| CDH2 | Cadherin 2, type 1 | X | | | Cell-cell adhesion. Functions |
| | | | | | during gastrulation and is |
| | | | | | required for establishment of |
| | | | | | left-right asymmetry |
| CDKN2A | Cyclin- | X | X | | Generates several |
| | dependaent | | | | transcription variants differing |
| | kinase inhibitor | | | | in their first exons |
| | 2A | | | | |
| CDKN2B | Cyclin- | | X | | Cell growth and cycle |
| | dependaent | | | | regulation |
| | kinase inhibitor | | | | |
| | 2B | | | | |
| COL9A1 | Collagen type IX, | | X | | Component of hyaline cartilage |
| | alpha 1 | | | | |
| CSNK1A1 | Casein kinase 1 | | X | | Use caseins as substrates |
| | aplha 1 | | | | |
| CTNNA1 | Catenin | X | | | Cellular differentiation |
| | (cadherin | | | | |
| | associated | | | | |
| | protein) alpha 1 | | | | |
| FGF13 | Fibroblast | | X | | Embryonic development, cell |
| | growth factor 13 | | | | growth, morphogenesis, tissue |
| | | | | | repair |
| FLT1 | FMS-related | | X | | Cell proliferation and |
| | tyrosine kinase | | | | differentiation |
| HDAC9 | Histone | X | X | | Transcription, cell cycle |
| | deacetylase 9 | | | | regulation and development. |
| | | | | | Histone acetylation/ |
| | | | | | deacetylation alters |
| | | | | | chromosome structure and |
| | | | | | transcription factor access to |
| | | | | | DNA |
| MYST4 | MYST histone | | X | | |
| | acetyltransferase 4 | | | | |
| PARD6B | Par-6 partitioning | X | X | | Membrane bound GTPase |
| | defective 6 | | | | |
| | homologue B | | | | |
| PARD6G | Par-6 partitioning | | X | | Filopodia formation |
| | defective 6 | | | | |
| | homologue G | | | | |
| POU5F1 | POU domain, | X | X | | Early developmental control |
| | class 5, | | | | |
| | tanscription | | | | |
| | factor 1 | | | | |
| PROM1 | Prominin 1 | | X | | Encodes a transmembrane |
| | | | | | glycoprotein |
| RBPSUH | Recombining | X | X | | Involved in notch signaling - |
| | binding protein | | | | cell-cell communication |
| | suppressor of | | | | determining cell fate |
| | hairless | | | | |
| | (Drosophila) | | | | |
| RPS27A | Ribosomal | X | X | | Produces ubiquitin and |
| | protein 27A | | | | ribosomal protein S27A |
| SLC2A1 | Solute carrier | X | | | Glucose transporter |
| | family 2, | | | | |
| | member 1 | | | | |
| SNAI2 | Snail homolog 2 | X | X | | Transcription factor |
| SOX1 | SRY box 1 | X | X | | Transcription factor involved in |
| | | | | | embryonic development |
| TP53 | Tumor protein 53 | X | | | Regulation of cell cycle |
| HITS | | 18 | 24 | 1 | |
| TOTAL | | | | | |

Modifications to these embodiments, further embodiments and modifications thereof will be apparent to the skilled person on reading this disclosure, and as such are within the scope of this invention. In particular, although the embodiments described above are for flat substrates, it will be apparent that the invention may also be applied to curved substrates, or substrates with irregular surfaces, e.g. macroscopic surfaces found in typical cultureware apparatus.

### REFERENCES

Dalby M J, Gadegaard N, Tare R, Andar A, Riehle M O, Herzyk P, Wilkinson C D W, Oreffo R O C. The control of human mesenchymal stem cell differentiation using nanoscale symmetry and disorder. Nature Materials 2007; 6:997-1003.
Denis F A, Hanarp P, Sutherland D S, Gold J, Mustin C, Rouxhet P G, et al. Protein Adsorption on Model Surfaces with Controlled Nanotopography and Chemistry. Langmuir 2002; 18(3):819-828.
Engler A J, Sen, S, Sweeney H L, Discher D E. Matrix Elasticity Directs Stem Cell Lineage Specification. Cell 2006; 126:677-689.
Gadegaard N, Dalby M J, Riehle M O, Wilkinson C D W. Optimizing substrate disorder for bone tissue engineering of mesenchymal stem cells. J. Vac. Sci. Technol. 2008; B 26(6) 2554-2557.
McFarlin D R, Finn K J, Murphy C J, Nealey P F. Embryonic stem cells do better on bumpy nanoscale mattress. News from The American Society for Cell Biology 46^{th} Annual Meeting, San Diego Calif., Dec. 9-13, 2006.
Mirmalek-Sani, S. H. et al., Characterization and Multipotentcy of Human Fetal Femur-Derived Cells—Implications for Skeletal Tissue Regeneration, Stem Cells 2006; 24, 1042-1053.
Oh S, Brammer K S, Li Y S J, Teng D, Engler A J, Chien S, Jin S. Stem cell fate dictated solely by altered nanotube dimension. PNAS 2009; Early Edition: 1-6
Oreffo R O, Bord S, Triffitt J T. Skeletal progenitor cells and ageing human populations. Clin Sci (Lond) 1998; 94(5):549-55.
Sutherland D S, Broberg M, Nygren H, Kasemo B. Influence of Nanoscale Surface Topography on the Functional Behaviour of an Absorbed Model Macromolecule. Macromolecular Bioscience 2001; 1(6):270-273.
Triffit, J. T. & Oreffo, R. O. in Advances in Organ Biology: Molecular and Cellular Biology of Bone (ed. Zardi, M.) 475-498 (JAI Press Inc., 1998)

1. A method for promoting the retention of a stem cell phenotype in a population of stem cells, the method comprising the steps of:
(i) providing a biocompatible substrate having an arrangement of topographical features arrayed in a pattern based on a notional symmetrical lattice in which the distance between nearest neighbour notional lattice points is between 10 nm and 10 μm, and wherein the topographical features are either located in register with the respective notional lattice points or are locally misordered such that the centre of each topographical feature is at most 10% of the distance between nearest neighbour notional lattice points from its respective notional lattice point;
(ii) providing a population of stem cells in contact with said arrangement of topographical features; and
(iii) culturing the population of stem cells under conditions that allow the stem cells to proliferate.

2. A method according to claim 1, wherein each topographical feature is at most 5% of the distance between nearest neighbour notional lattice points from its respective notional lattice point.

3. A method according to claim 1, wherein the topographical features of the biocompatible substrate are recesses into and/or protrusions from the surface of the substrate.

4. A method according to claim 1, wherein the distance between nearest neighbour notional lattice points is at least 50 nm.

5. A method according to claim 1, wherein the distance between nearest neighbour notional lattice points is at most 5 μm.

6. A method according to claim 1, wherein the height or depth of the topographical features is at least 5% of the distance between nearest neighbour notional lattice points from the remainder of the surface of the substrate.

7. A method according to claim 1, wherein the topographical features are cylindrical pits or pillars, hemi-spherical pits or pillars, or part-spherical pits or pillars.

8. A method according to claim 7, wherein the diameter of the topographical features is at least 20 nm.

9. A method according to claim 1, wherein the notional symmetrical lattice is a square, rectangular, hexagonal, parallelogram, rhombic, or trigonal lattice.

10. A method according to claim 1, wherein within an area of the substrate within which the arrangement of topographical features is formed, the topographical features account for between 5% and 35% of the surface of the substrate.

11. A method according to claim 1, wherein the stem cells are adult stem cells, such as mesenchymal stem cells, neural stem cells, haemopoietic stem cells, endothelial stem cells, or adipose-derived stem cells.

12. A method according to claim 1, wherein the stem cells are embryonic stem cells.

13. A method according to claim 1, wherein the stem cells are cultured in step (c) for more than 5 days.

14. A method according to claim 1, wherein the stem cells are passaged at least once during step (c).

15. Cultureware for promoting the retention of a stem cell phenotype, said cultureware including a biocompatible substrate having an arrangement of topographical features arrayed in a pattern based on a notional symmetrical lattice in which the distance between nearest neighbour notional lattice points is between 10 nm and 10 μm, and wherein the topographical features are either located in register with the respective notional lattice points or are locally misordered such that the centre of each topographical feature is at most 10% of the distance between nearest neighbour notional lattice points from its respective notional lattice point.

16. Cultureware according to claim 16, wherein each topographical feature is at most 5% of the distance between nearest neighbour notional lattice points from its respective notional lattice point.

17. Cultureware according to claim 15, wherein the topographical features of the biocompatible substrate are recesses into and/or protrusions from the surface of the substrate.

18. Cultureware according to claim 15, wherein the distance between nearest neighbour notional lattice points is at least 100 nm.

19. Cultureware according to claim 15, wherein the distance between nearest neighbour notional lattice points is at most 3 μm.

20. Cultureware according to claim 15, wherein the height or depth of the topographical features is at least 5% of the distance between nearest neighbour notional lattice points from the remainder of the surface of the substrate.

21. Cultureware according to claim 15, wherein the topographical features are cylindrical pits or pillars, hemi-spherical pits or pillars, or part-spherical pits or pillars.

22. Cultureware according to claim 15, wherein the diameter of the topographical features is at least 20 nm.

23. Cultureware according to claim 15, wherein the notional symmetrical lattice is a square, rectangular, hexagonal, parallelogram, rhombic, or trigonal lattice.

24. Cultureware according to claim 15, wherein within an area of the substrate within which the arrangement of topographical features is formed, the topographical features account for between 5% and 35% of the surface of the substrate.

25. Cultureware according to claim 15, wherein the cultureware is a culture flask, petri dish, plate or coverslip.

26. Cultureware according to claim 15 having a population of stem cells stored on said biocompatible surface.

27. Cultureware according to claim 26, wherein the cultureware is a cell seeded construct or conduit.
